(19) United States
(12) Patent Application Publication
(10) Pub. No.: US 2013/0165685 A1
(43) Pub. Date: Jun. 27, 2013

(54) SYNTHESIS OF OMEGA-AMINO CARBOXYLIC ACIDS AND THEIR ESTERS FROM UNSATURATED FATTY ACID DERIVATIVES

(71) Applicants: Peter Hannen, Herten (DE); Harald Haeger, Luedinghausen (DE); Martin Roos, Haltern am See (DE)

(72) Inventors: Peter Hannen, Herten (DE); Harald Haeger, Luedinghausen (DE); Martin Roos, Haltern am See (DE)

(73) Assignee: EVONIK DEGUSSA GmbH, Essen (DE)

(21) Appl. No.: 13/806,555

(22) PCT Filed: Dec. 7, 2010
(86) PCT No.: PCT/EP2010/069039

§ 371 Date: Mar. 11, 2013

(30) Foreign Application Priority Data
Jun. 25, 2010 (DE) 10 2010 026 196.3

(51) Int. Cl. C07C 227/08

(52) U.S. Cl. CPC C07C 227/08
USPC 560/155

(57) ABSTRACT

The invention relates to a process for preparing omega-amino acids or their esters, which is characterized by the following steps:
c) ozonolysis of unsaturated fatty acids or fatty acid derivatives,
d) reductive amination of the reaction mixture obtained from the reaction with ozone, to give the omega-amino acid or ester thereof,
   the reaction being carried out with a C₁-C₅ alcohol in a mixture with at least 0.5% by weight of water as solvent, based on the total amount of solvent.

The invention relates to a process for preparing omega-amino acids or their esters by ozonolysis and subsequent reductive amination. A further subject of the invention is a process for preparing fatty amines by ozonolysis of unsaturated fatty acids or fatty acid derivatives and subsequent reductive amination.

Ozonolysis for the purposes of the invention means the cleaving of a carbon-carbon double bond by exposure to ozone. Depending on the manner of workup, carbonyl compounds, alcohols or carboxylic acids are obtained.

The reaction takes place by 1,3-dipolar cycloaddition of ozone onto a C,C double bond of an olefin (1) to form the primary ozonide (1,2,3-trioxolane, 2). The radical R denotes hydrogen, an alkyl, alkylene or alkynyl group or an aryl group. The radicals R in one molecule may be the same or different and are optionally substituted. This compound (2) is an unstable intermediate which immediately breaks down into an aldehyde fragment (3) and a carbonyl oxide (4). The compounds are shown in the scheme below:

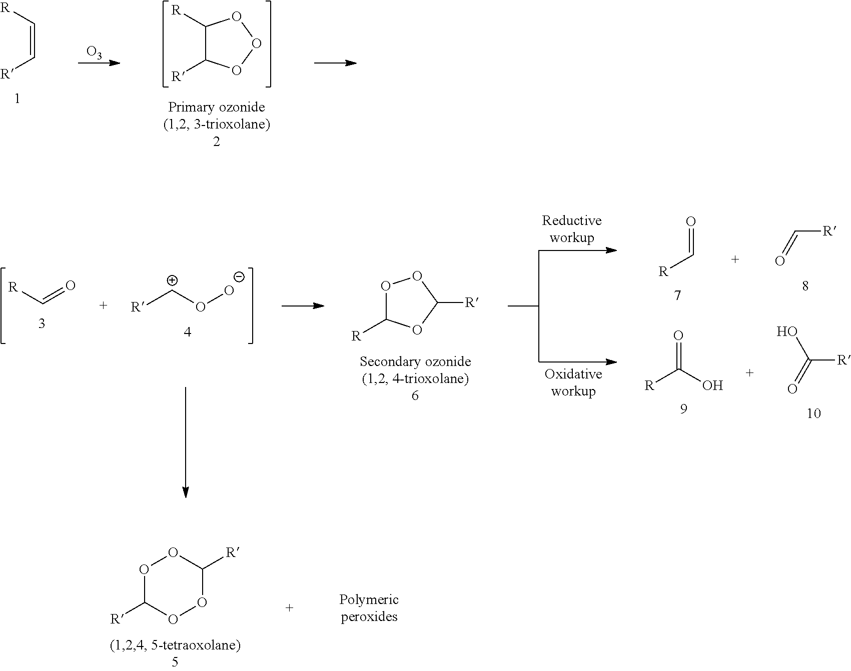

The carbonyl oxide may on the one hand undergo polymerization or dimerization to form a 1,2,4,5-tetraoxolane (5), or may recombine in a further cycloaddition to form a secondary ozonide (1,2,4-trioxolane, 6). Starting from compound 6, aldehydes (7, 8) can be prepared via a reductive workup, or carboxylic acids (9, 10) via an oxidative workup. The aldehydes in turn can be reduced further to the alcohol.

A key drawback of this reaction sequence is the formation of the usually explosive secondary ozonides, polymeric peroxides and/or 1,2,4,5-tetraoxolanes, some of which are stable compounds and might therefore accumulate in downstream reaction steps or workup steps, and pose a considerable hazard. Furthermore, in the case of oxidative or reductive workup of secondary ozonides, it is necessary to use an oxidation or reduction equivalent, respectively, such as, for example, dimethyl sulphide, triphenylphosphine, etc. For this reason, reaction in an industrially implementable process on the large scale, at economically acceptable cost, is difficult.

In order to avoid the formation of secondary ozonides or relatively high molecular mass ozonide adducts, the prior art describes the scavenging of the carbonyl oxide (4) by means of a nucleophile, such an alcohol, for example. In most cases the nucleophile is also the solvent. Recombination with the carbonyl group of the second cleavage product (3) to form the secondary ozonide is prevented in this way (S. L. Schreiber et al. Tet. Lett. 1982, 23 (38), 3867; R. E. Claus, S. L. Schreiber Organic Syntheses, Coll. Vol. 7, 1990, 168).

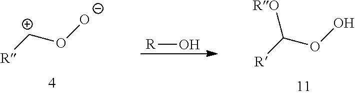

In certain cases, in a subsequent step, the hydroperoxide group (11) is acetylated and subjected to decomposition under basic catalysis, and a carboxylic ester (13) of the alcohol used is formed.

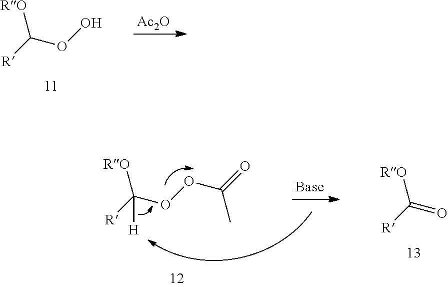

Other works use a carboxylic acid as solvent for the same purpose (DE 22 07 699 A1, DE 24 33 408 A1, DE 30 37 487 A1). The carboxyl group of the carboxylic acid undergoes addition onto the carbonyl oxide 4. The workup of the hydroperoxide derivative (14) takes place in turn as described. However, the mixed anhydride (15) that is formed in the course of the basic cleavage must further be cleaved with water, with heating, to form the free acid (16).

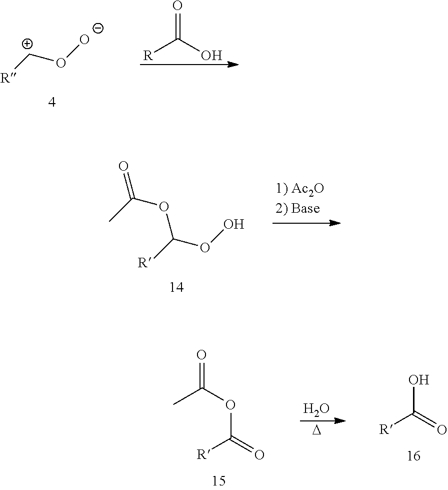

In the case of the ozonolysis of asymmetrical olefins such as methyl oleate, for example, there are, according to the existing viewpoint, two possibilities for the opening of the primary ozonide. Shown below are the different reaction pathways for the ozonolysis of methyl oleate in acetic acid as protic solvent.

Following addition of ozone onto the double bond, the primary ozonide 18 can be cleaved at position 5 (pathway a) or at position 4 (pathway b) of the 1,2,3-trioxolane. The respective carbonyl oxide intermediate is scavenged by the acetic acid. In the subsequent workup step, the hydroperoxide group of the compounds 20 and 24 is acetylated with acetic anhydride. Since there are now a comparatively good leaving group (acetate group) and an acidic proton, simple addition of sodium acetate as base is accompanied by deprotonation, with elimination of the acetate group, thus initially forming from compounds 21 and 25, the anhydrides of 22 and 26. Following cleavage of these anhydrides with water, the corresponding monocarboxylic acid and monomethyl dicarboxylate, respectively, are obtained.

The result, therefore, is a statistical distribution of the four anticipated products. Since effective control of the reaction pathways is not possible, this approach is most unsuitable for the synthesis of, for example, methyl 9-oxononanoate, from which the desired 9-aminononanoic acid and/or its esters are obtainable via a reductive amination.

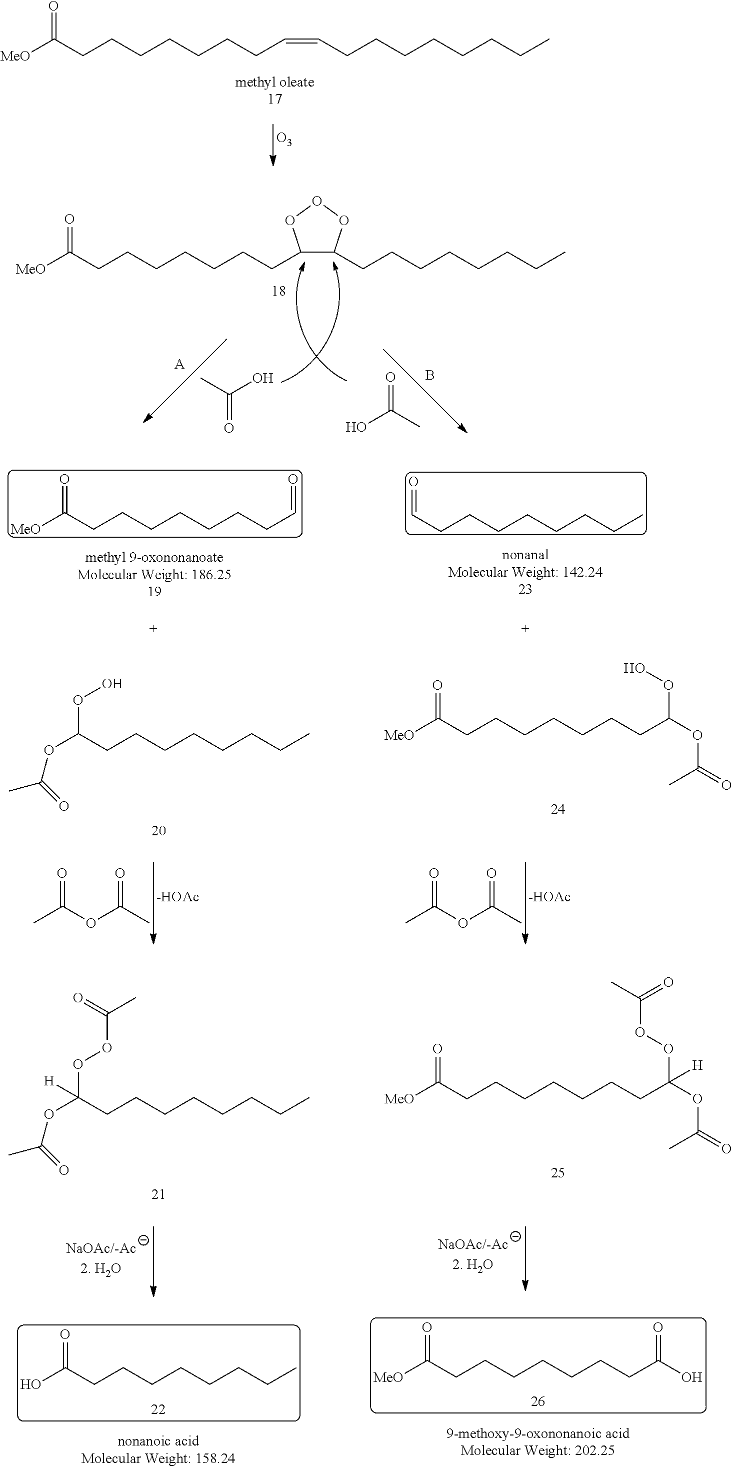

One approach as a solution to this problem, in other words the preparation of omega-oxocarboxylic acids and their esters, which avoids the formation of secondary ozonides lies in ozonolysis in the presence of NMMO (N-methylmorpholine N-oxide) as catalyst, as described for other systems by Dussault et al. (P. H. Dussault et al., Org. Lett. 2006, 8 (15), 3199). A disadvantage, however, is that for the ozonolysis of methyl oleate it is necessary to use three equivalents of NMMO in order to achieve a satisfactory outcome here.

A reaction regime which, in contrast, is technically relevant for the ozonolysis of unsaturated fatty acids and direct recovery of the aldehydes lies in the use of a mixture of acetone with water (around 5%) as solvent. In the experiments described by Dussault, however, only terminal olefins were used (P. H. Dussault, C. E. Schiaffo, J. Org. Chem. 2008, 73, 4688).

DE 34 40 620 A1 describes the effect of water in the ozonolysis of fatty acid derivatives. The observation was made that, in the presence of water in the reaction mixture, aldehydes are formed during the ozonolysis itself, and not only during the reductive cleavage of the ozonides. However, increased yields of aldehydes were described only on reductive workup with hydrogen and a metal catalyst. In that case the water was added preferably only in the reduction step. As a result, there is still the problem of the formation of ozonides in the ozonolysis stage.

The above-described ozonolysis processes have the disadvantage that they are not compatible with the conditions of the reductive amination and do not reliably avoid the formation of explosive ozonides in the ozonolysis stage.

Moreover, many solvents used in the ozonolysis, such as carboxylic acids and ketones, for example, are unsuitable for use in the reductive amination, since they lead to the formation of by-products.

It was the technical object of the invention, therefore, to provide a process for preparing omega-amino acids or their esters that on the one hand avoids the formation of ozonides and on the other hand allows a direct conversion of the reaction product from the ozonolysis in the reductive amination.

This technical object is achieved by a process for preparing omega-amino acids or their esters, which is characterized by the following steps:
a) ozonolysis of unsaturated fatty acids or fatty acid derivatives,
b) reductive amination of the reaction mixture obtained from the reaction with ozone, to give the omega-amino acid or ester thereof,
   the reaction being carried out with a C₁-C₅ alcohol in a mixture with at least 0.5% by weight of water as solvent, based on the total amount of solvent.

Ozonolysis for the purposes of the invention means the reaction of a fatty acid or a fatty acid derivative with ozone.

It has surprisingly been found that the process carried out in this way allows safer implementation in comparison to the conventional processes of the prior art. Ozonides and the carbonyl oxide formed as an intermediate react directly with the water present. The adduct of ozonide and water undergoes immediate decomposition to form a carbonyl group and hydrogen peroxide. Hence there is no formation of the hazardous secondary ozonides or oligomeric or polymeric ozonides, all of which would initially be formed in the established processes for the reductive workup of intermediates of the ozonolysis by means of hydrogen and metal catalysts or complex metal hydrides. A further advantage of the process is that the aldehydes are obtained exclusively in one reaction step.

It has been found that in the case of the use of C₁-C₅ alcohols as solvent in a mixture with at least 0.5% by weight of water, the reaction product of the ozonolysis, without separation or workup, can be supplied directly to a reductive amination and that in this way it is possible to prepare omega-amino acids with high yields. Hence the process of the invention shows a simple and safe pathway for preparing, from unsaturated fatty acid esters, corresponding omega-aminocarboxylic acids and also fatty amines.

In one particular embodiment the solvent contains 1% to 20% by weight, preferably 2% to 15% by weight and more preferably 5% to 10% by weight of water, based on the total amount of solvent.

Fatty acid or fatty acid derivatives used are those having at least one double bond. Especially preferred fatty acids and fatty acid derivatives are compounds selected from the group consisting of oleic acid, alkyl oleates, undecylenic acid, alkyl undecylenates, erucic acid and alkyl erucates.

As starting products for the process of the invention it is also possible, however, to use other unsaturated fatty acids or fatty acid derivatives. These include, for example, myristoleic acid, palmitoleic acid, petroselinic acid, elaidic acid, vaccenic acid, gadoleic acid, icosenoic acid, cetoleic acid and nervonic acid and their esters. These are monounsaturated fatty acids. Furthermore, it is also possible to use polyunsaturated fatty acids such as, for example, linoleic acid, linolenic acid, calendic acid, punicic acid, elaeostearic acid, arachidonic acid, timnodonic acid, clupanodonic acid and cervonic acid or their esters.

In a further preferred embodiment, the ozonolysis and the reductive amination take place directly after one another without isolation or workup of the reaction mixture from the ozonolysis.

Particularly preferred solvents used are a secondary or tertiary alcohol, very preferably 2-propanol or tert-butanol.

The ozonolysis is typically carried out in alcohol as solvent. The reaction mixture further contains at least 0.5% by weight of water, based on the total amount of solvent. The unsaturated fatty acid ester is present typically at a concentration of 0.1 to 1 mol/l. If higher concentrations of fatty acids are used, care should be taken to ensure that the amount of water added is selected to be always at least stoichiometric relative to the number of reacted double bonds. The ozonolysis is carried out preferably at temperatures of 0 to 25° C. Ozone generation is typically done using an ozone generator. The feed gas used by this ozone generator is industrial air or a mixture of carbon dioxide and oxygen. In the ozone generator, the ozone is prepared by silent electrical discharge. This forms oxygen radicals which react with oxygen molecules to form ozone.

After the ozonolysis has been carried out, the resulting reaction mixture is supplied, without further workup or isolation, to the reductive amination. This reductive amination is preferably carried out with the aid of a Raney nickel catalyst and hydrogen. This reductive amination is known per se in the prior art and takes place in accordance with the customary process parameters. With preference, the pressure during the reductive amination is in the range from 30 to 100 bar, preferably 50 to 100 bar, and the temperature is in the range from 50 to 150° C.

In the reductive amination it is preferred to supply hydrogen to the reaction product from the ozonolysis. For this purpose the reaction mixture from the ozonolysis is transferred to an autoclave and charged with the catalyst. After the autoclave has been closed, ammonia is added under pressure, and hydrogen. The reaction mixture is heated and, after the reaction has been carried out, the autoclave is let down and the reaction products are worked up. The reaction forms fatty amines and also omega-amino acids or their esters in high yields.

The advantage of the process of the invention is that it avoids the formation of explosive by-products such as secondary ozonides or else oligomeric ozonides in the ozonolysis as a result of addition of water. Moreover, in the case of the process of the invention, the direct formation of aldehydes takes place in one reaction step without use of further reduction equivalents such as, for example, hydrogen/catalyst, complex metal hydrides, dimethyl sulphide, triphenylphosphine, zinc/acetic acid, as is necessary in the prior art. Since the reaction mixture of the ozonolysis can be reacted further immediately in the reductive amination, workup steps are avoided and hence it is also possible to increase the overall yield and to have a reaction regime which overall is far less expensive. The process of the invention also makes it possible to carry out a reductive amination of the reaction mixture from the ozonolysis directly.

The example below is intended to illustrate the invention.

### EXAMPLE

Methyl oleate (4 g, 95% by weight purity, 0.012 mol) is charged to a two-necked flask with gas inlet tube in a solvent mixture of tert-butanol (20 mL) and water (1 mL, 0.056 mol). The feed gas, consisting of 5% by volume oxygen in carbon dioxide, is passed through the ozone generator at a flow rate of 40 mL/min. The ozone generator used is an apparatus from Anseros of the type ‘COM-AD’. The ozone generator is set at maximum output. The ozone-containing gas mixture is passed into the reaction mixture with thorough stirring. The outgoing gas stream is passed via gas wash bottles into an aqueous potassium iodide solution with a strength of approximately 5% by weight. After 60 minutes, the substrate has been converted, and the introduction of gas is halted. According to analysis by GC, the reaction mixture contains 39.5% by weight of 9-nonanal and 38.2% by weight of methyl 9-oxononanoate.

The reaction mixture is introduced into a 100 mL steel autoclave and charged with Raney nickel (1.2 g). After the autoclave has been closed, ammonia (11.35 g, 0.67 mol) is added via a pressurized cylinder. 70 bar of hydrogen are injected, and heating is carried out to 80° C. After six hours, the reaction mixture is cooled and the autoclave is let down. According to analysis by GC, the aldehydes have been completely converted. This has taken place with formation of 46.4% by weight of 9-aminononane and 24.0% by weight of methyl 9-aminononanoate.

1. A process for preparing an omega-amino compound, the process comprising:
ozonolysis of an unsaturated fatty acid or fatty acid derivative, to obtain an ozonolysis reaction mixture; and
reductive amination of the ozonolysis reaction mixture to obtain an omega-amino compound,
wherein the ozonolysis occurs in the presence of a C1 to C5 alcohol in a mixture comprising at least 0.5% by weight of water, based on a total amount of solvent in the mixture.

2. The process according to claim 1, wherein the mixture comprises 1% to 20% by weight of the water, based on the total amount of solvent.

3. The process according to claim 1, wherein the mixture comprises 2% to 15% by weight of the water, based on the total amount of solvent.

4. The process according to claim 1, wherein the mixture comprises 5% to 10% by weight of the water, based on the total amount of solvent, with the proviso that the water is present at least in a stoichiometric amount relative to a number of double bonds reacted.

5. The process according to claim 1 4, comprising ozonization of a fatty acid or fatty acid derivative comprising at least one double bond.

6. The process according to claim 1, wherein the ozonolysis and the reductive amination are occur directly after one another without isolation or workup of the ozonolysis reaction mixture.

7. The process according to claim 1, wherein the unsaturated fatty acid or fatty acid derivative is at least one selected from the group consisting of oleic acid, an alkyl oleate, undecylenic acid, an alkyl undecylenate, erucic acid, and an alkyl erucate.

8. The process according to claim 1, wherein the reductive amination occurs in the presence of hydrogen and a catalyst.

9. The process according to claim 8, wherein the catalyst is Raney nickel.

10. The process according to claim 1, wherein the mixture comprises a secondary or tertiary alcohol.

11. The process according to claim 1, wherein the mixture comprises 2-propanol or tert-butanol.

12. The process according to claim 1, wherein the reductive amination occurs at a pressure of 30 to 100 bar.

13. The process according to claim 1, wherein the reductive amination occurs at a temperature of 50 to 150° C.

14. The process according to claim 1, which is suitable for preparing fatty amines.

15. The process according to claim 2, wherein the mixture comprises 2% to 15% by weight of the water, based on the total amount of solvent.

16. The process according to claim 1, wherein the reductive amination occurs at a pressure of 50 to 100 bar.
